(19) United States
(12) Patent Application Publication
(10) Pub. No.: US 2024/0350440 A1
(43) Pub. Date: Oct. 24, 2024

(54) EXTRACTION OF ABSCISIC ACID FROM STRAWBERRIES

(71) Applicant: KING FAISAL UNIVERSITY, Al-Ahsa (SA)

(72) Inventors: HANY MOHAMED ABD EL-LATEEF AHMED, AL-AHSA (SA); MAI MOSTAFA KHALAF ALI, AL-AHSA (SA); AHMED MOHAMMED ABU-DIEF MOHAMMED, AL-AHSA (SA); HAZEM MOHAMED SHAHEEN, AL-AHSA (SA); GABER ELSABER ABD EL-WANIS BATIHA, AL-AHSA (SA); MANAL ALY SHALABY, AL-AHSA (SA); RASHA MANSOUR NAGI, AL-AHSA (SA)

(21) Appl. No.: 18/222,720

(22) Filed: Jul. 17, 2023

Related U.S. Application Data
(62) Division of application No. 18/137,094, filed on Apr. 20, 2023, now abandoned.

(51) Int. Cl. A61K 31/192; A23B 7/024; A61P 9/10

(52) U.S. Cl. CPC A61K 31/192; A23B 7/024; A61P 9/10

(57) ABSTRACT

The disclosure of the present patent application relates generally to the extraction and purification of abscisic acid (ABA) from strawberries and its application in medicine and disease treatment in mammals and specifically humans. The extraction and purification process uses gel column chromatography to provide a highly pure composition of ABA, and the application of such includes treating heart disease.

### BACKGROUND

### 1. Field

The disclosure of the present patent application relates generally to the extraction and purification of abscisic acid from strawberries and its application in medicine and disease treatment in mammals and specifically humans.

### 2. Description of the Related Art

Abscisic acid (ABA) is a phytohormone that affects plant ripening and has recently been considered in human disease treatment. ABA is commonly named the ‘stress hormone’ and regulates many aspects of plant growth and development. More recently, ABA has received considerable attention in mammalian cells and has been seen to be linked to human disease. This phytohormone is present in all plants, vegetables, and fruits, such as strawberries.

Abscisic acid (ABA) is a plant hormone which was discovered at the beginning of the 1960s. Said acid regulates important functions in plants, such as the response to water and nutrient scarcity, ultraviolet radiation, seed dormancy and germination, and root growth. Natural ABA consists of a mixture of two isomers. ABA is synthesized in leaves, stems, roots and green fruits. However, it was not until the discovery of the biosynthesis thereof in animal cells that interest has been raised in elucidating possible parallels between its role in plant systems and animals.

Extracting ABA from plant tissue is the first step in a qualitative and quantitative analysis. At present, the main extraction method is to use 80% methanol solution to extract overnight, but the extraction method has a long extraction time, which generally takes 18-23 hours. The extraction steps are complicated, and too many operations affect the recovery rate of ABA in addition to other shortcomings. Therefore, the overall efficiency of extracting ABA by methanol extraction is not high, so improving the extraction steps is one of the important research contents for fine analysis of ABA. There are many methods for the determination of plant endogenous hormones, and in recent years, high performance liquid chromatography (HPLC) is more commonly used. Ultra performance liquid chromatography (UPLC) is a newer liquid chromatography technology based on HPLC. It has higher separation speed and sensitivity than HPLC. It is currently mainly used in proteomics and drug analysis, environmental monitoring and many other high-end fields.

There have also been efforts to optimize a simple extraction method and validate LC-MS quantification of ABA as well as the identification and quantification of ABA derivatives (PA, DPA, and ABA-GE) in strawberry fruit. Hormone extraction was performed using either methanol (80% v/v-S1) or acetone:water:acetic acid (80:19:1 v/v-S2) solutions with or without the use of sonication. Additionally, there have been efforts for simple, reliable and rapid method of extracting and partially purifying the phytohormone (+)-abscisic acid and its catabolites for liquid chromatography-electrospray ionization tandem mass spectrometry (LC-ESI-MS-MS) analysis. Solvents were optimized for only partial purification of abscisic acid and related compounds by solid-phase extraction using Oasis HLB cartridges. The eluted metabolites were then analyzed by LC-MS-MS. But there has been no purification method or method for disease treatment in mammals or specifically humans reported involving abscisic acid.

Thus, a method for preparing an extract of abscisic acid from plant materials which can be sufficiently pure and safe to be used in pharmaceutical, nutritional and cosmetic compositions, and a method of using such pure extract, are desired.

### SUMMARY

The present subject matter relates to the extraction and purification of abscisic acid from certain fruits, including by way of non-limiting example, strawberries, and its application in medicine and disease treatment in patients, such as mammals including humans.

The present subject matter provides a method for effective extraction and purification of abscisic acid (ABA) from strawberries, as well as highly purified ABA for further use of said purified ABA in applications in fields such as medicine for human disease treatment.

In one embodiment, the present subject matter relates to a process for obtaining purified abscisic acid (ABA) comprising: providing a fruit; washing and freeze-drying the fruit to obtain a lyophilized fruit; grinding the lyophilized fruit to obtain a ground lyophilized fruit; dissolving the ground lyophilized fruit in methanol 80% to form a solution; centrifuging the solution multiple times and obtaining a supernatant of the solution; filtering the supernatant of the solution to obtain a filtered supernatant; passing the filtered supernatant through a silica gel glass column to extract, fraction and purify all components from the filtered supernatant to obtained fractioned components according to a molecular weight of each respective component; collecting the fractioned components in multiple test tubes; analyzing an amount of abscisic acid in each of the multiple test tubes using High-Performance Liquid Chromatography (HPLC); and selecting those test tubes of the multiple test tubes containing a high level of abscisic acid to obtain a composition having a high abscisic acid purity.

In another embodiment, the present processes result in a purified ABA of about 97 to about 98.8% purity.

In a further embodiment, the present subject matter relates to a pharmaceutically acceptable composition comprising a therapeutically effective amount of an abscisic acid composition as described herein and a pharmaceutically acceptable carrier.

In one embodiment, the present subject matter relates to a method of treating a disease, disorder, or condition in a patient, comprising administering to a patient in need thereof a therapeutically effective amount of the pharmaceutically acceptable composition as described herein.

In still another embodiment, the present subject matter relates to a composition comprising abscisic acid (ABA), wherein the abscisic acid has a purity of about 97 to about 98.8%. In this regard, the abscisic acid can be powdered abscisic acid, and can be provided with a carrier suitable for injection to a human.

These and other features of the present subject matter will become readily apparent upon further review of the following specification.

### BRIEF DESCRIPTION OF THE DRAWINGS

FIG. 1 shows an image of a heart showing nearly normal heart muscle striation (StainH&EX200) (grade 1).

FIG. 2 shows an image of a heart showing severe muscle fibers necrosis (gray arrow), severe cardiac fibrosis (black arrow) with aggregation of lymphocytes (White arrow) (grade 4).

FIG. 3 shows an image of a heart having standard ABA administered, showing the degree of muscle necrosis with myofibroblasts proliferation (thin arrow) and cardiac fibrosis (thick arrow) (grade 3).

FIG. 4 shows an image of a heart having the ABA compositions obtained herein administered thereto, showing muscle necrosis with deposition of collagen in between cardiac muscle (arrow) (grade 3).

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

“Subject” as used herein refers to any animal classified as a mammal, including humans, domestic and farm animals, and zoo, sports, and pet companion animals such as household pets and other domesticated animals such as, but not limited to, cattle, sheep, ferrets, swine, horses, poultry, rabbits, goats, dogs, cats and the like.

“Patient” as used herein refers to a subject in need of treatment of a condition, disorder, or disease.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The present subject matter relates to the extraction and purification of abscisic acid (ABA) from fruits and its application in medicine and disease treatment. More specifically, it provides a method for effective extraction and purification of the ABA compound from fruits, as well as a highly purified ABA material for further use n applications as a pharmaceutical or medicament in mammal and human disease treatment. ABA is sensitive and fragile and must be carefully treated for successful extraction and purification.

In one embodiment, the present subject matter relates to a process for obtaining purified abscisic acid (ABA) comprising: providing a fruit; washing and freeze-drying the fruit to obtain a lyophilized fruit; grinding the lyophilized fruit to obtain a ground lyophilized fruit; dissolving the ground lyophilized fruit in methanol 80% to form a solution; centrifuging the solution multiple times and obtaining a supernatant of the solution; filtering the supernatant of the solution to obtain a filtered supernatant; passing the filtered supernatant through a silica gel glass column to extract, fraction and purify all components from the filtered supernatant to obtained fractioned components according to a molecular weight of each respective component; collecting the fractioned components in multiple test tubes; analyzing an amount of abscisic acid in each of the multiple test tubes using High-Performance Liquid Chromatography (HPLC); and selecting those test tubes of the multiple test tubes containing a high level of abscisic acid to obtain a composition having a high abscisic acid purity.

In another embodiment, the present processes result in a purified ABA of about 97 to about 98.8% purity.

In an embodiment, the fruit used in the present processes can be selected from the group consisting of strawberries, figs, cranberries, blueberries, apricots, bananas, oranges, lemon, grapefruit, and combinations thereof. In one embodiment in this regard, the fruit used in the present processes is strawberries.

In an embodiment, a gel filtration method can be used, whereby the step of passing the filtered supernatant through a silica gel glass column involves letting all components from the filtered supernatant descend by gravity through a silica gel glass column, whereby the fractioned components are converted to layers through the silica gel glass column according to the molecular weight of each respective component. This technique can separate all components and fraction the solution coming from the tap at the end of the silica gel glass column into test tubes. All test tubes can be numbered and submitted for HPLC analysis to identify which test tube(s) contained a high level of ABA. By this technique, ABA having a purity of about 97 to about 98.8% can be obtained, which ABA can be further prepared for human injection in disease treatment.

In an embodiment of the present processes, the solution can be centrifuged two, three, four, five, or more times. In one embodiment in this regard, the solution can be centrifuged three times.

In another embodiment, the composition having a high abscisic acid purity produced by the present methods is an abscisic acid powder.

The present methods can result in an abscisic acid powder composition, having an abscisic acid purity of about 97 to about 98.8%.

In another embodiment, filtered purified ABA of about 97-98.8% purity is obtained.

In an embodiment, purified abscisic acid (ABA) of about 97-98.8% purity is used for treatment of certain diseases in mammals including humans including heart disease.

In another embodiment, the present subject matter relates to a pharmaceutically acceptable composition comprising a therapeutically effective amount of the abscisic acid powder composition as described herein and a pharmaceutically acceptable carrier, excipient, or vehicle. In certain embodiments in this regard, the pharmaceutically acceptable carrier can be suitable for injection to a human.

In some embodiments, the present compositions can be used for combination therapy, where other therapeutic and/or prophylactic ingredients can be administered therewith.

Non-limiting examples of suitable excipients, carriers, or vehicles useful herein include liquids such as water, saline, glycerol, polyethylene glycol, hyaluronic acid, ethanol, and the like. A thorough discussion of pharmaceutically acceptable excipients and salts useful herein is available in Remington's Pharmaceutical Sciences, 18th Edition. Easton, Pa., Mack Publishing Company, 1990, the entire contents of which are incorporated by reference herein.

The present compounds are typically administered at a therapeutically or pharmaceutically effective dosage, e.g., a dosage sufficient to provide treatment for a disease, disorder, or condition as described herein. Administration of the compounds or pharmaceutical compositions thereof can be by any method that delivers the compounds systemically and/or locally. These methods include oral routes, parenteral routes, injection routes, intraduodenal routes, and the like. In an embodiment, the present methods involve injection routes of administration.

While human dosage levels have yet to be optimized for the present compounds, generally, a daily dose is from about 0.01 to 10.0 mg/kg of body weight, for example about 0.1 to 5.0 mg/kg of body weight. The precise effective amount will vary from subject to subject and will depend upon the species, age, the subject's size and health, the nature and extent of the condition being treated, recommendations of the treating physician, and the therapeutics or combination of therapeutics selected for administration. The subject may be administered as many doses as is required to reduce and/or alleviate the signs, symptoms, or causes of the disease or disorder in question, or bring about any other desired alteration of a biological system.

In employing the present compounds for treatment of a disease, disorder, or condition herein, any pharmaceutically acceptable mode of administration can be used with other pharmaceutically acceptable excipients, including solid, semi-solid, liquid or aerosol dosage forms, such as, for example, tablets, capsules, powders, liquids, suspensions, suppositories, aerosols or the like. The present compounds can also be administered in sustained or controlled release dosage forms, including depot injections, osmotic pumps, pills, transdermal (including electrotransport) patches, and the like, for the prolonged administration of the compound at a predetermined rate, preferably in unit dosage forms suitable for single administration of precise dosages.

The present compounds may also be administered as compositions prepared as foods for foods or animals, including medical foods, functional food, special nutrition foods and dietary supplements. A “medical food” is a product prescribed by a physician that is intended for the specific dietary management of a disorder or health condition for which distinctive nutritional requirements exist and may include formulations fed through a feeding tube (referred to as enteral administration or gavage administration).

A “dietary supplement” shall mean a product that is intended to supplement the human diet and may be provided in the form of a pill, capsule, tablet, or like formulation. By way of non-limiting example, a dietary supplement may include one or more of the following dietary ingredients: vitamins, minerals, herbs, botanicals, amino acids, and dietary substances intended to supplement the diet by increasing total dietary intake, or a concentrate, metabolite, constituent, extract, or combinations of these ingredients, not intended as a conventional food or as the sole item of a meal or diet. Dietary supplements may also be incorporated into foodstuffs, such as functional foods designed to promote control of glucose levels. A “functional food” is an ordinary food that has one or more components or ingredients incorporated into it to give a specific medical or physiological benefit, other than a purely nutritional effect. “Special nutrition food” means ingredients designed for a particular diet related to conditions or to support treatment of nutritional deficiencies.

Liquid pharmaceutically administrable compositions can, for example, be prepared by dissolving, dispersing, etc. an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, sodium acetate, sodium citrate, cyclodextrine derivatives, sorbitan monolaurate, triethanolamine acetate, triethanolamine oleate, etc.

Another manner of administration is parenteral administration, generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents, solubility enhancers, and the like, such as for example, sodium acetate, sorbitan monolaurate, triethanolamine oleate, cyclodextrins, etc.

Another approach for parenteral administration employs the implantation of a slow-release or sustained-release system, such that a constant level of dosage is maintained. The percentage of active compound contained in such parenteral compositions is highly dependent on the specific nature thereof, as well as the activity of the compound and the needs of the subject.

### Methods of Use:

The present subject matter involving ABA has valuable pharmaceutical properties, which makes it commercially utilizable. Accordingly, the present subject matter further relates to a method of treating a disease, disorder, or condition in a patient, comprising administering to a patient in need thereof a therapeutically effective amount of the pharmaceutically acceptable composition as described herein.

In this regard, in certain embodiments, the disease, disorder, or condition can be selected from the group consisting of myocardial infarction, diabetes, inflammatory intestinal disorders, atherosclerosis, cancer, insulin resistance, hyperglycemia, glucose intolerance, prediabetes, inflammation, heart disease, and combinations thereof. In one specific embodiment in this regard, the disease, disorder, or condition can be myocardial infarction.

In other embodiments, the patient can be a mammal, such as a human.

The following examples relate to obtaining, testing, and/or analyzing abscisic acid as described herein.

### EXAMPLES

### Example 1

Strawberry fruits were purchased from a neighboring farm then washed, blotted, and freeze-dried, and the pedicel was removed. The strawberries were lyophilized well and ground. Then the lyophilized ground strawberries were dissolved in methanol 80% to form a solution, and the solution was centrifuged 3 times. The supernatant of the centrifuged solution was filtered. Column chromatography was then used in the abscisic acid (ABA) purification. After fractioning of the filtered supernatant solution into test tubes, the amount of abscisic acid in each tube was analyzed by using High-Performance Liquid Chromatography (HPLC). A material composition of about 97-98.8% purity ABA was obtained.

The fraction of strawberry components obtained by the previously disclosed silica gel column gravity method according to their molecular weights were analyzed using HPLC. The following samples from two test tubes contained a high level of ABA.

TABLE 1
The HPLC results of ABA samples

| | Ret Time | Area | | Amount | |
|---|---|---|---|---|---|
| Name | [min] | [mAU*s] | Amt/Area | [mg/ml] | Totals |
| Std | 1.949 | 2070.9011 | 1.07296e−5 | 2.22200e−2 | 2.22200e−2 |
| S1 | 1.949 | 9212.6845 | 1.07296e−5 | 9.88487e−2 | 9.88497e−2 |
| S2 | 1.945 | 9064.7334 | 1.07296e−5 | 9.72612e−2 | 9.72612e−2 |

This table illustrates the results of standard ABA, sample 1 (S1), and sample2 (S2).

### Example 2

The extracted and purified ABA was tested on an albino rat after inducing myocardial infarction disease by using a high dose of adrenaline at Alexandria faculty of medicine and all tests of examination were performed (chemical, electrocardiograph Histological).

All parameters indicate that ABA can be used in treating Myocardial infarction.

TABLE 2
Analyses of oxidative stress markers and antioxidant enzymes

| | | | | Catalase |
|---|---|---|---|---|
| | MDA | NO | GSH | (U/g |
| Group | (μmol/g protein) | (μM/g protein) | (μmol/g protein) | protein) |
| Control normal | 3.2 ± 0.00^{e} | 35 ± 0^{c} | 19.3 ± 0^{a} | 176 ± 0^{a} |
| MI | 11.13 ± 0.3^{a} | 103.06 ± 3.05^{a} | 14.29 ± 0.13^{b} | 64 ± 0.58^{c} |
| Standard ABA | 6.13 ± 1.34^{bc} | 48.61 ± 5.61^{bc} | 20.01 ± 2.27^{a} | 112 ± 23.09^{abc} |
| Extracted ABA | 5.68 ± 0.27^{bcd} | 47.78 ± 4.49^{bc} | 21.66 ± 0.39^{a} | 97 ± 1.73^{bc} |

TABLE 3
Analyses of biological cardiac markers

| | LDH | CK-MB | Troponin |
|---|---|---|---|
| Group | (U/L) | (U/L) | (ng/ml) |
| Control normal | 442.00 ± 64.00^{c} | 0.75 ± 0.05^{d} | 1.05 ± 0.15^{e} |
| MI | 978.00 ± 22^{.00a} | 1.46 ± 0.06^{a} | 2.2 ± 0.1^{a} |
| Standard ABA | 587.33 ± 30.12^{b} | 1.22 ± 0.03^{b} | 1.72 ± 0.05^{b} |
| Extracted ABA | 482.67 ± 4.63^{bc} | 1.14 ± 0.02^{bc} | 1.71 ± 0.02^{b} |

TABLE 4
Parameters of electrocardiogram assessment.

| | carotid flow | | | Stroke volume | | |
|---|---|---|---|---|---|---|
| | mean | HR (mean | COP (mean | (mean | ST | RR(interval/ |
| Group | (ml/min) | b/min) | ml/min) | ml/min) | height(mm) | sec) |
| Control/normal | 23.72 ± 0.04^{a} | 239 ± 1.37^{d} | 23.82 ± 0.05^{a} | 0.1 ± 0.001^{a} | 21.25 ± 0.43^{d} | 0.75 ± 0.004^{a} |
| MI | 10.65 ± 0.04^{c} | 267 ± 1.24^{ab} | 10.75 ± 0.03^{c} | 0.04 ± 0^{c} | 50.87 ± 0.99^{a} | 0.67 ± 0.003^{d} |
| Standard ABA | 17.4 ± 0.16^{b} | 255.77 ± 1.23^{bc} | 17.52 ± 0.1^{b} | 0.069 ± 0.001^{b} | 39.33 ± 2.15^{b} | 0.7 ± 0.003^{c} |
| Extracted ABA | 18.06 ± 1.15^{b} | 247.4 ± 9.69^{cd} | 18.57 ± 1.04^{b} | 0.077 ± 0.009^{b} | 42.98 ± 4.05^{b} | 0.73 ± 0.034^{b} |

### Example 3

With reference to the Figures, the histopathological assessment of H&E-stained (hematoxylin and eosin stained) heart tissue was scored according to severity of myocardial changes: grade 1 (normal histological the appearance of the myocardium), grade 2 (focal necrosis of myocardium), grade 3 (focal necrosis of myocardial fiber with neutrophil infiltration and interstitial edema), and grade 4 (extensive necrosis of myocardial fiber with hemorrhage, interstitial edema, marked neutrophil infiltration and severe myofibrillary degeneration).

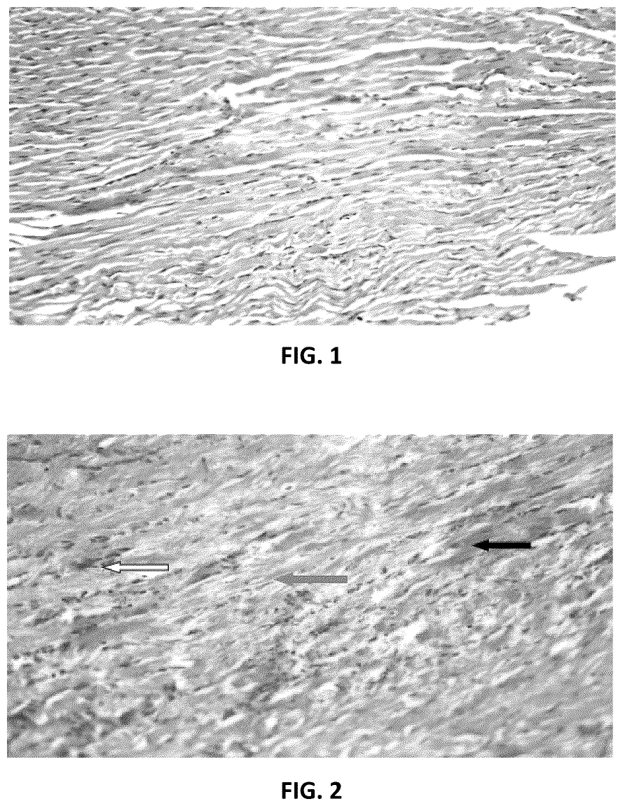

FIG. 1 shows the normal control group, with a heart showing nearly normal heart muscle striation (StainH&EX200) (grade 1).

FIG. 2 shows the myocardial infarction (MI) group, with a heart showing severe muscle fiber necrosis (grey arrow) and severe cardiac fibrosis (black arrow), with aggregation of lymphocytes (white arrow) (grade 4).

FIG. 3 shows the group treated with standard ABA, with a heart showing the degree of muscle necrosis with myofibroblasts proliferation (thin arrow) and cardiac fibrosis (thick arrow) (grade 3).

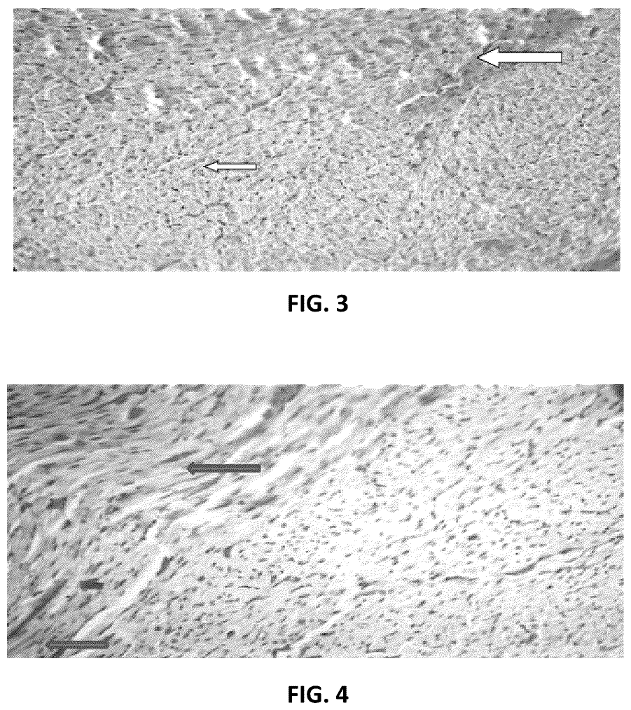

FIG. 4 shows the group treated with the present extracted ABA, where the heart shows muscle necrosis with deposition of collagen in between cardiac muscle (arrow) (grade 3).

It is to be understood that the abscisic acid (ABA) material, use/application, and properties thereof are not limited to the specific embodiments or examples described above, but encompasses any and all embodiments within the scope of the generic language of the following claims enabled by the embodiments described herein, or otherwise shown in the drawings or described above in terms sufficient to enable one of ordinary skill in the art to make and use the instant subject matter compounds.

1. A process for obtaining purified abscisic acid (ABA) comprising:
providing a fruit;
washing and freeze-drying the fruit to obtain a lyophilized fruit;
grinding the lyophilized fruit to obtain a ground lyophilized fruit;
dissolving the ground lyophilized fruit in methanol 80% to form a solution;
centrifuging the solution multiple times and obtaining a supernatant of the solution;
filtering the supernatant of the solution to obtain a filtered supernatant;
passing the filtered supernatant through a silica gel glass column to extract, fraction and purify all components from the filtered supernatant to obtained fractioned components according to a molecular weight of each respective component;
collecting the fractioned components in multiple test tubes;
analyzing an amount of abscisic acid in each of the multiple test tubes using High-Performance Liquid Chromatography (HPLC); and
selecting those test tubes of the multiple test tubes containing a high level of abscisic acid to obtain a composition having a high abscisic acid purity,
wherein the high abscisic acid purity is about 97 to about 98.8% and the high level of abscisic acid is from 9.7612e-2 to 9.8847e-2 mg/ml.

2. The process of claim 1 wherein the fruit is strawberries.

3. (canceled)

4. The process of claim 1, wherein the solution is centrifuged three times.

5. (canceled)

6. The process of claim 1, wherein the step of passing the filtered supernatant through a silica gel glass column comprises letting all components from the filtered supernatant descend by gravity through the silica gel glass column, whereby the fractioned components are converted to layers through the silica gel glass column according to the molecular weight of each respective component.

7-17. (canceled)
